(19) United States
(12) Plant Patent Application Publication
(10) Pub. No.: US 2003/0226182 P1
(43) Pub. Date: Dec. 4, 2003

(54) Variegated illicium 'Shady Lady'

(76) Inventors: Thomas H. Dodd III, Mobile, AL (US); Thayer Dodd, Mobile, AL (US)

Correspondence Address: ALSTON & BIRD LLP, BANK OF AMERICA PLAZA, 101 SOUTH TRYON STREET, SUITE 4000, CHARLOTTE, NC, 28280-4000, US

(21) Appl. No.: 10/156,357

(22) Filed: May 28, 2002

(51) Int. Cl. A01H 5/00

(52) U.S. Cl. PLT/226

(57) ABSTRACT

Illicium floridanum forma variegatum, a new and distinct form of Illicium floridanum is presented. It differs from typical I. floridanum in that its flowers are pink and its leaves are variegated. The flower color and variegation are persistent in greenhouse-grown offshoots of the original plant.

### BACKGROUND OF THE INVENTION

The present invention relates to a new and distinct form of Illicium floridanum,referred to commercially as a variegated Illicium, and given the cultivar name “Shady Lady”.

This invention has produced a new variegated-leaved form of Illicium floridanum f. variegatum. It was propagated by shoot from I. floridanum forma albiflorum “Semmes,” a white-flowered nonvariegated-leaved form of I. floridanum. Illicium floridanum J. Ellis forma variegatum Gaddy “Shady Lady” is an Illicium having variegated leaves and a light pink flower from the form of Illicium floridanum (star-anise, Florida anise).

This invention has not been observed under all possible environmental conditions. The phenotype may vary significantly with variations in environment such as temperature, light intensity, and day length, without, however, any variation in genotype

This new variety is endowed with a unique combination of traits which are determined to be the basic characteristics of this invention which in combination distinguish it as new and distinct, and materially contributes to its attractiveness. Among its important and dominant characteristics are the following: (1) the leaves of the new variety are variegated having a gray-green color at the exterior edges of the leaf and ranging from to a yellow-green to yellow at the interior of the leaf; and (2) the flowers of the new variety are light pink with the inner petals fading to a softer pink.

This invention is best compared to wild floridanum. A comparison for the leaf and flower characteristics is shown in Table I wherein it can be seen that the leaves both as to size and shape are distinct. Also, the flower color is distinct from the wild floridanum.

The flower color and leaf variegation of forma variegatum has remained stable in offshoots from the original variegated-leaved plant which have been grown at Carolina Nurseries in Moncks Corner, S.C., shows that the foregoing characteristics come true to form and are established and transmitted through succeeding propagation.

### BRIEF DESCRIPTION OF THE DRAWINGS

The accompanying photographs show specimens of the new variety, with their foliage and flowers in different stages of development.

FIG. 1 is a close-up photograph which illustrates the variegated leaves of the new plant;

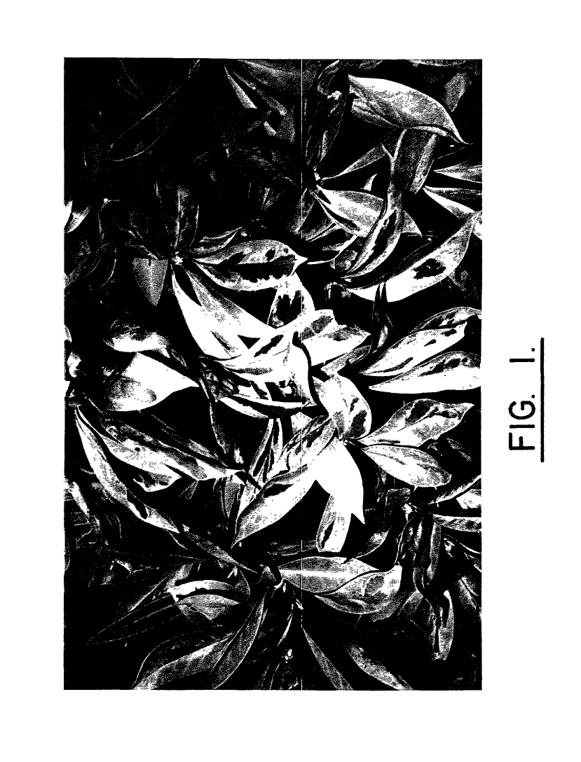

FIG. 2 is a photograph which illustrates a cluster of buds of the “Shady Lady” illicium;

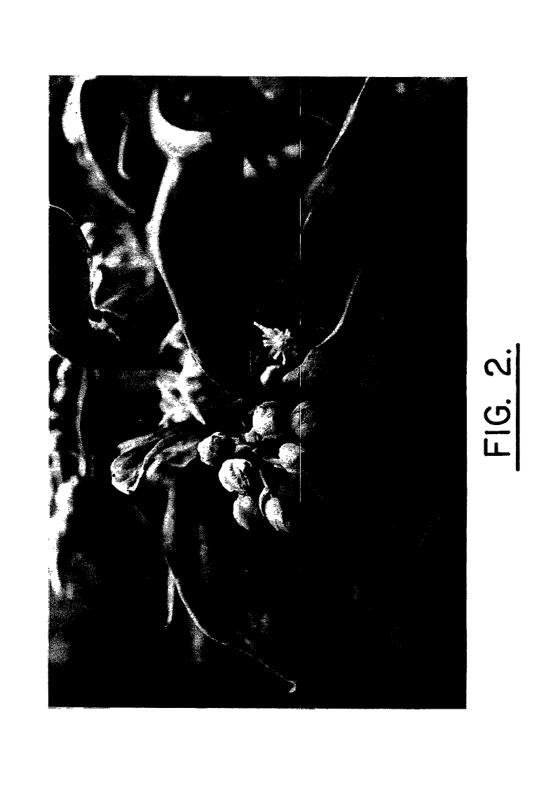

FIG. 3 is a photograph which illustrates a single flower of the new plant; and

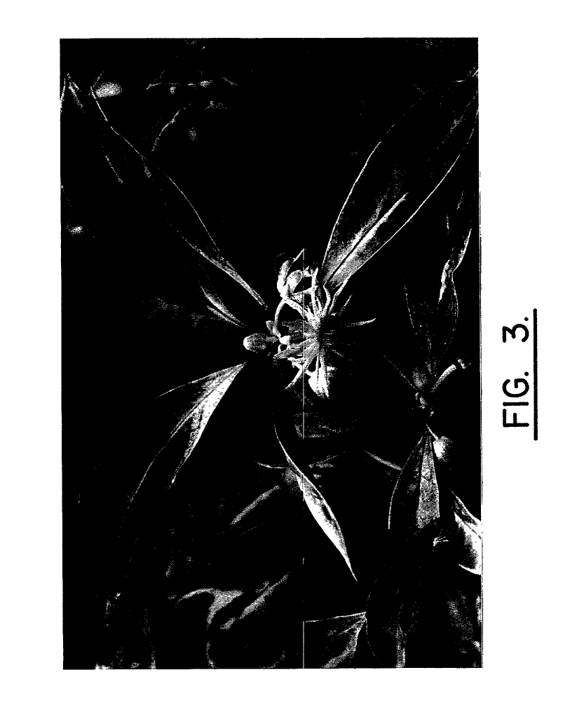

FIG. 4 is a photograph showing the “Shady Lady” illicium.

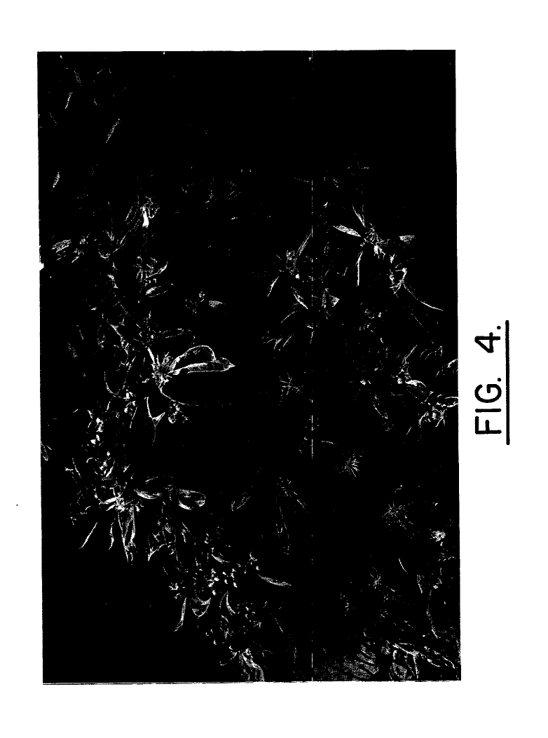

The photographs of the drawing were taken Apr. 28, 1999 in Semmes, Ala. and illustrate the typical foliage form, with color as true as possible in this type of color photograph.

### DETAILED DESCRIPTION OF THE INVENTION

The following is a detailed description of the invention based on plants produced under commercial practices in the Carolina Nurseries greenhouse in Moncks Corner, S.C. in August, 1998. Color references are made in accordance with The Royal Horticultural Colour Chart of the British Color Council, except where general color terms of ordinary dictionary significance are obvious.

### LATIN DIAGNOSIS

Differt a Illicium floridanum J. Ellis foliis variegatis et floribus roseus. (Differs from Illicium floridanum J. Ellis by its variegated leaves and by its pink flowers.)

Illicium floridanum J. Ellis forma variegatum Gaddy “Shady Lady” is a pink-flowered, variegated-leaved form of Illicium floridanum (star-anise, Florida anise). Table 1 gives data on various taxonomic characters of the new form and typical floridanum. Characters seen in young plants of I. floridanum f. variegatum not mentioned in the description of typical I. flordianum (Vincent in Flora of North America, 1997) include asymmetrical leaves, curved leaf tips (apices), pink flower color, and leaf variegation. In container-grown plants less than 0.5 meter tall, leaves were significantly smaller than those of typical I. floridanum; however, this difference may be due to the fact that these plants are young shoots.

Here, it is asserted that Illicium floridanum f. variegatum is a pink-flowered, variegated-leaved form of I. floridanum. It was propagated by shoot from I. floridanum forma albiflorum “Semmes,” a white-flowered nonvariegated-leaved form of I. floridanum. It, however, has pink flowers and variegated leaves.

The flower color and leaf variegation of forma variegatum has remained stable in offshoots from the original variegated-leaved plant which have been grown at Carolina Nurseries in Moncks Corner, S.C.

### THE PLANT

Origin: Dodd & Dodd Nursery Post Office Drawer 439 Semmes, Ala. 36575

Parentage: Branch sport of Illicium floridanum ‘Alba’, Variety ‘Semmes’.

Type: Evergreen. Leaves mostly elliptic to lanceolate in shape. Apex acute to acuminate with leaf base cuneate.

Commercial class: Ornamental.

Classification: Broad-leaf evergreen.

Propagation: This plant has been asexually reproduced at Carolina Nurseries by means of semi-hardwood cuttings. All rooted cuttings have remained true to the new variety.

Form: Upright rounded shrub. Multi-trunked.

Height: 6 feet to 8 feet. Slightly shorter than type.

Stem color: Brownish gray, dotted with small lenticels.

Foliage:

Arrangement.—Alternate. In pseudo-whorls at end of stem.

Leaf size.—about 6.5-11 cm long and 1.3-2.5 cm wide.

Leaf shape.—often asymmetrical. Entire.

Texture.—leathery.

Aspect.—Smooth. Aromatic anise-like odor.

Color.—variegated with the primary green of the leaves being a grey-green — Royal Horticultural Society 189A; variegated colors range from grey-green (RHS 188B) to yellow-green (RHS 144B) to yellow (RHS 3D), often with an admixture of the latter three colors.

### THE BUD

Size: Small.

Shape: Globular with pointed tips. Rose-pink (RHS-60D).

Rate of opening:

Color: Rose-pink (RHS-60D).

### THE FLOWER

Locality where grown: Moncks Corner, S.C.

Blooming habit: Has started to flower as early as late February in Moncks Corner and continued until mid-April. Peak times have been April 1^{st}, with a tendency to repeat in summer and fall.

Continuity: Continuous.

Borne: Singly on a ½″-1½″ pedicel.

Shape: Perfect, 1″-1½″ in diameter.

Fragrance: Reminiscent of a wet dog or fish.

Tepalage: 20-30 strap-shaped petals, soft pink, ligulate. Pollinated by a variety of insects.

Number.—1.

Arrangement.—Single.

Shape.—Round.

Texture.—Thick, leathery.

Aspect.—Waxy.

Size.—1″-1½″ in diameter.

Color.—Light pink (RHS-66D) fading to softer pink (RHS-69C).

Quality of bloom: Abundant; outdoors.

Peduncle: Length. — ½″-1½″.

### THE REPRODUCTIVE ORGANS

Stamens: Number. — 25-50. Anthers before dehiscence.

Pistil: Number — 11-21.

Fruit: One seeded, dehiseent follicles, 11-15 arranged in a whorl, ¾″-1″ diameter, first green, then yellow, and finally brown, maturing in November. Has a star-like configuration, distinct, seeds BB-like, brown and shiney.

TABLE 1
Illicium floridanum forma variegatum “Shady Lady”
and its relationship to wild floridanum.¹

| | | “Shady Lady” |
|---|---|---|
| | Wild floridanum | forma variegatum |
| LEAF LENGTH | 5-21 cm | 6.5-11 cm |
| LEAF WIDTH | 1.5-6 cm | 1.3-2.5 cm |
| LEAF SHAPE | Elliptic to lanceolate | Often asymmetrical |
| LEAF BASE | Cuneate | Narrowly cuneate |
| LEAF APEX | Acute to acuminate | Often curved |
| LEAF THICKNESS | Thin to leathery | Leathery |
| LEAF COLOR | Olive | Variegated |
| FLOWER COLOR | Maroon | Pink |

That which is claimed:

1. A new and distinct form of Illicium floridanum J. Ellis Illicium floridanum forma variegatum “Shady Lady” distinguished from typical I. floridanum by its variegated leaves and pink flowers.
